SEQUENCE LISTING (53 sequences)

SEQ ID NO: 1 - protein, 887 aa, Bifidobacterium bifidum
  REGION 1..887: note = SEQ ID NO: 1
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKT

SEQ ID NO: 2 - protein, 965 aa, Bifidobacterium bifidum
  REGION 1..965: note = SEQ ID NO: 2
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGAL

SEQ ID NO: 3 - protein, 1038 aa, Bifidobacterium bifidum
  REGION 1..1038: note = SEQ ID NO: 3
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQ

SEQ ID NO: 4 - protein, 1142 aa, Bifidobacterium bifidum
  REGION 1..1142: note = SEQ ID NO: 4
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQI

SEQ ID NO: 5 - protein, 1211 aa, Bifidobacterium bifidum
  REGION 1..1211: note = SEQ ID NO: 5
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTAT

SEQ ID NO: 6 - protein, 1296 aa, Bifidobacterium bifidum
  REGION 1..1296: note = SEQ ID NO: 6
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEF

SEQ ID NO: 7 - protein, 1304 aa, synthetic construct
  REGION 1..1304: note = SEQ ID NO: 7
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSERVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 8 - protein, 1931 aa, synthetic construct
  REGION 1..1931: note = Synthetic construct
MKKPLGKIVASTALLISVAFSSSIASAAVEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDNALKALTDAYAGLTEKTPEPAPVSKSELDKKIKAIEAEKLDGSKYTAESWKAFETALAHAKAVIASDSATQQNVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGSALSKTGASVAIIASVAAAMLAVGAGVMALRRKRS

SEQ ID NO: 9 - protein, 1341 aa, synthetic construct
  REGION 1..1341: note = Synthetic construct
MKKPLGKIVASTALLISVAFSSSIASAIEDATRSDSTTQMSSTPEVAYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFSATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEQRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVRVTVTNADTTTPSGVVCAGLTEIELKTATSKFVANTSAALSSLTVNGTKVSDSVLAAGSYNTPAIIADVKAEGEGNASVTVLPAHDNVIRVITESEDHVTRKTFTINLGTEQEFPADSDERDQHQHQHQHQQ

SEQ ID NO: 10 - protein, 1752 aa, synthetic construct
  REGION 1..1752: note = Synthetic construct
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 11 - protein, 1935 aa, synthetic construct
  REGION 1..1935: note = Artificial Sequence
MAVRRLGGRIVAFAATVALSIPLGLLTNSAWAVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTKAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGADKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVDWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTAGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQLNYDDDSTGTVTWNTQTAGDFTFKPALDAKLKVTDKTVTVTYQGKSAVIDITVSQPAPTVSKTDLDKAIKAIEAKNPDSSKYTADSWKTFADAMAHAKAVIADDSATQQDVDKALKALTDAYAGLTEKTPEPAPVSKSELDKKIKAIEAEKLDGSKYTAESWKAFETALAHAKAVIASDSATQQDVDAALGALTSARDGLTEKGEVKPDPKPEPGTVDKAALDKAVKKVEAEKLDGSKYTADSWKAFETALAHAKAVIGNANSTQFDIDNALSMLNDARAALKEKPGRIIAIIDGGALSKTGASVAIIASVAAAMKAVGAGVMALRPPKW

SEQ ID NO: 12 - protein, 887 aa, Bifidobacterium bifidum; identical to SEQ ID NO: 1
  REGION 1..887: note = >SEQ ID NO: 12

SEQ ID NO: 13 - protein, 965 aa, Bifidobacterium bifidum; identical to SEQ ID NO: 2
  REGION 1..965: note = >SEQ ID NO: 13

SEQ ID NO: 14 - protein, 1038 aa, Bifidobacterium bifidum; identical to SEQ ID NO: 3
  REGION 1..1038: note = >SEQ ID NO: 14

SEQ ID NO: 15 - protein, 1142 aa, Bifidobacterium bifidum; identical to SEQ ID NO: 4
  REGION 1..1142: note = >SEQ ID NO: 15

SEQ ID NO: 16 - protein, 1211 aa, Bifidobacterium bifidum; identical to SEQ ID NO: 5
  REGION 1..1211: note = >SEQ ID NO: 16

SEQ ID NO: 17 - protein, 1296 aa, Bifidobacterium bifidum; identical to SEQ ID NO: 6
  REGION 1..1296: note = >SEQ ID NO: 17

SEQ ID NO: 18 - protein, 1720 aa, Bifidobacterium bifidum
  REGION 1..1720: note = >SEQ ID NO: 18
VEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGDVTMNLTTKVANDTEAAANITLKQTVFPKGGKTDAAIGTVTTASKSIAAGASADVTSTITAASPKLWSIKNPNLYTVRTEVLNGGKVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQAIAGDNAVLGGDKDETWAKFDLTSTINRDRNAPSVIMWSLGNEMMEGISGSVSGFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNNVPVVVYTDAAKVKLYFTPKGSTEKRLIGEKSFTKKTTAAGYTYQVYEGSDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERDYPAADMTVTVGSEQTSGTATEGPKKFAVDGNTSTYWHSNWTPTTVNDLWIAFELQKPTKLDALRYLPRPAGSKNGSVTEYKVQVSDDGTNWTDAGSGTWTTDYGWKLAEFNQPVTTKHVRLKAVHTYADSGNDKFMSASEIRLRKAVDTTDISGATVTVPAKLTVDRVDADHPATFATKDVTVTLGDATLRYGVDYLLDYAGNTAVGKATVTVRGIDKYSGTVAKTFTIELKNAPAPEPTLTSVSVKTKPSKLTYVVGDAFDPAGLVLQHDRQADRPPQPLVGEQADERGLTCGTRCDRVEQLRKHENREAHRTGLDHLEFVGAADGAVGEQATFKVHVHADQGDGRHDDADERDIDPHVPVDHAVGELARAACHHVIGLRVDTHRLKASGFQIPADDMAEIDRITGFHRFERHVG

SEQ ID NO: 19 - protein, 1049 aa, Bifidobacterium adolescentis
  REGION 1..1049: note = >SEQ ID No: 19
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNSADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIVVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 20 - protein, 625 aa, Lactobacillus sakei
  REGION 1..625: note = >SEQ ID No: 20
MQPNIQWLDTPAVFRVGQLPAHSDHRYYATLAEMAQQQSSFEQSLNGTWQFHYSVNAASRPKSFYELAFDAQDFEPITVPQHIELAGYEQLHYINTMYPWEGHYYRRPAFSTSDDKQHLGMFSEADYNPVGSYLHHFDLTPALRNQRVIIRFEGVEQAMYVWLNGQFIGYAEDSFTPSEFDLTPYLKETDNCLAVEVHKRSSAAFIEDQDFFRFFGIFRDVKLLAKPRTHLEDLWVIPEYDVVQQTGQVKLRLQFSGDENRVHLRIRDQHQIILTADLTSAAQVNGLYKMPELVQAWSNQTPNLYTLELEVVDQAGETIEISQQPFGFRKIEIKDKVMLLNGKRLVINGVNRHEWHPETGRTITAEDEAWDIACMQRNHINAVRTSHYPDRLSFYNGCDQAGIYMMAETNLESHGSWQKMGAVEPSWNVPGSYDEWEAATLDRARTNFETFKNHVSILFWSLGNESYAGSVLEKMNAYYKQQDPTRLVHYEGVFRAPEYKATISDVESRMYATPAEIKAYLDNAPQKPFILCEYMHDMGNSLGGMQSYIDLLSQYDMYQGGFIWDFIDQALLVTDPVTGQRELRYGGDFDDRPSDYEFSGDGLVFATRDEKPAMQEVRYYYGEHK

SEQ ID NO: 21 - protein, 330 aa, Lactobacillus sakei
  REGION 1..330: note = >SEQ ID No: 21
MKNQQCRRLDTIMANTNKRLAVIFGDVTLGLKGPDFHYLFSYQTGGPESLRIQGKEWLYRSPKPTFWRATTDNDRGNQFPLKSGMWLAADQFIACQSITVAIDGQTIPLPIAPENNRYSGQETAQEVTVTYTYQTITTPQTTVEVSYTIQASGKIRVAVTYHGQAGLPSLPVFGLRFVMPTPATRFIYQGLSGETYPDRMAGGMAGEYEVTGLPVTPYLVPQDCGVHMATDWVTIYRQAVLDNRLREPVETGLKFKMVDQPFAFSCLPYTAEELENATHHSELPAPHRTVLSLLGAVRGVGGIDSWGSDVEAAYQIDATQDHHLEFEISF

SEQ ID NO: 22 - protein, 1049 aa, Bifidobacterium adolescentis
  REGION 1..1049: note = >SEQ ID No: 22
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEGACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 23 - protein, 626 aa, Lactobacillus amylovorus
  REGION 1..626: note = >SEQ ID No: 23
MKANIKWLDDPEVFRINQLPAHSDHPFYKDYREWQNHSSSFKQSLNGAWQFHFSKDPQSRPIDFYKRSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGTPDGGMGSYIKLIDKYPQYMGGFIWDFIDQALLVHDPVSGQDVLRYGGDFDDRHSDYEFSGDGLMFADRTPKPAMQEVRYYYGLHK

SEQ ID NO: 24 - protein, 316 aa, Lactobacillus amylovorus
  REGION 1..316: note = >SEQ ID No: 24
MAYTNNLHVVYGEASLGVNGQDFAYLFSYERGGLESLKIKDKEWLYRTPTPTFWRATTDNDRGSGFNQKAAQWLGADMFTKCVGIHVQVDDHRFDELPVAPINNQFSNQEFAHEVKVAFDYETLTTPATKVKIIYNINDFGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 25 - protein, 1052 aa, Bifidobacterium bifidum
  REGION 1..1052: note = >SEQ ID No: 25
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALRDKNGTIVWRTATKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHQLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDEEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYTIEAARRHEDLPKPRHNYLRLLAAQMGVGGDDSWGAPVHTAYQLPAGRPLTLDVNLELI

SEQ ID NO: 26 - protein, 1052 aa, Bifidobacterium bifidum
  REGION 1..1052: note = >SEQ ID No: 26
MNTTDDQRKNGDPIVSPSIPTTAWLADPRVYAVHRLDAHSDHACWSRSPVDGESTDLRQSLDGEWRVRVETAPTGRFPDGTSDGPDWISDVSPLFAAPGFDDSSFSRVQVPSHLETAGLLAPQYVNVQYPWDGHEDPKAPAIPEHGHVAVYRREFDADGEVAQAVREGRPVTLTFQGAATAIYVWLNGSFIGYAEDSFTPSEFDVTDAIKVDGNVLAVACYEYSSASWLEDQDFWRLHGLFRSVELNARPAAHVADLHADADWDLATSRGSLSLDVLIDGAANAATADFALWDKNGTIVWHIVTKADGTLHAEAEIDDAAPWSAERPDLYELSVTLLDADGKVLETARTRIGFRHVAIEDGILKLNGKRLVFRGVNRHEFDCRRGRAITEEDMLWDIRFMKRHNINAVRTSHYPNQSRWYELCDEYGIYLIDETNLETHGSWNSPGDIPVGTSVPGDDEAWLGACIDRLDSMILRDRNHPSVLVWSLGNESYAGEVLKAMSAHAHRLDPGRPVHYEGVNWNHAYDGISDFESRMYAKPAEIQDWLEHGDERGEASKPFVSCEYMHAMGNSCGGLSEFIDLERYERYSGGFIWDYIDQGLVQRLPDGSERLSVGGEWGDRPTDYEFVGNGIVFADRTPSPKAQEVKQLYSPVKLAPDGHGVTIENRNLFAGTDGYVFAARLLEDGHEIWHADYRFDVAAGDTQHHDIAFPDIDADGDTREVTYEVDLLLAEATAWAPAGYELAFGQLTGTLNPEQDITETSHDDDGRATRTLSRWNAGIRRDDKEILLSRTQGGIVSWKRDDREMVIRRPELVTFRPLTDNDRGNHSGFDRAAWFAAGRYAIVTETKIHESDDGLVAEYQYELADPNHTPVSVTYHVNSDMRMQLTVEYPGNATDMASLPAFGIEWELPGEYDRLRYYGPGPEETYRDRKQGGKLGIWDATAKASMAPYLMVQETGSHEDVRWLEATDIQGHGLRVTQRGDRHFTASLLPWNTYMIEAARRHEDLPEPRHNYLRLLAAQMGVGGDDSWGAPVHTAYQLPAGRPLTLDVNLELI

SEQ ID NO: 27 - protein, 1055 aa, Bifidobacterium breve
  REGION 1..1055: note = >SEQ ID No: 27
MTNSMQGKAKTIMTNLQSAQQFSQAWLTDPRVFAVNRLAAHSSHKFYDHSPQCGEAMDLKQSLDGQWRVQMLDLADLADNELAEAAFAQPGYDAAGFSPIEVPSALETKGFLNHQYVNQQYPWSGHESPVAPDVPKHNHVALYRHEFSLEPKAAAVLEANKTAADDAAKRRVTLTFQGAATAIVVWLNGAFIGYAEDSFTPSEFDVTDVLRDGVNTLAVACFEFSSASWLEDQDFWRLHGIFRSVELEAQPLVHVNDLRVLADYDHTTGEGSLDVVALLRNAGTAAAVAATVLDAAGNTVWHSKLTAGADAETLTVKANVGKVNPWSAEEPTLYTLQVVATDAAGQVIEAALQRIGFRHFAIEDGLMKLNGKRIVFKGVDRHEFDARTGRTIAEADMIEDIHSFKRLNINAVRTSHYPNETRWYELCDEYGIYVLDETNLETHGSWTDPGDVFQPARAIPGSKDEWRAACVDRTASMVRRDYNHPSVVIWSLGNEAFGGDVFYSMRDFVHENDPFRPVHYEGTFNDPEFSAATDIMSRMYAKPDEIVKLYLGEDGKKPYISCEYSHSMGNSTGGLHLYTELERYPLYQGGFIWDYVDQALWQDCGNGTERLAYGGDFEDRPNDYEFSGDGVMFADRTPSPKAQEVKQLYANVKLVPDESGVTITNDNLFISTASSLFTARVLVDGAERWHANYRFDVPAGETVREPIAFPKVTDLVALSGSAEVTYEVDQRLAEATDWAPAGYELTFGQYVAAVSFDDGAADAVVAGDAEVAADGFNAGIHTDFGEVLLSKTQGGMVSFKRDGREMVIRRPNLTTFRALTDNDRGNGSGFERAQWMAAGRYARVTGTSVEETADGKGLKATYSYELADAKHTPVTVHYEVDAALRVHLTVEYPGEADAATLPAFGLEWILPKQYDRLRFYGLGPEETYADRLHGAKLGVFSRTAAEDCAPYLLPQETGNHEQVRWAEITDEYGHGMRVTAAGGTRFATSLLPYSSLMFEDALHQNELPKPRHTFLRLLAAQMGVGGDDTWGAPVHDEFQVPADQPLKLDVTLELI

SEQ ID NO: 28 - protein, 689 aa, Bifidobacterium catenulatum
  REGION 1..689: note = >SEQ ID No: 28
MTQRRSYRWPQPLAGQQARIWYGGDYNPDQWPEEVWDDDVRLMKKAGVNLVSVGIFSWAKIETSEGVYDFDWLDRIIDKLGEAGIAVDLASATASPPMWLTQAHPEVLWKDYRGDVCQPGARQHWRPTSPVFREYALKLCRAMAEHYKGNPYVVAWHVSNEYGCHNRFDYSEDAERAFRKWCEERYGTIDAVNDAWGTAFWAQRMNDFTEIVPPRFIGDGNFMNPGKLLDFKRFSSDALKAFYVAERDALAEITPDLPLTTNFMVSAAGSVLDYDDWGREVDFVSNDHYFIPGEAHLDELAFSASLVDGIARKDPWFLMEHSTSAVNWRPVNYRKEPGQLVRDSLAHVAMGADAVCYFQWRQSKAGAEKFHSAMVPHTGEDSAVFRDVCELGADLNTLADNGLLGTKLAKSKVAVVFDYESEWATEHTATPTQKVHHVDEPLQWFRALADHGVTADVVPVSSNWDEYEVVVLPSVYILSEETTRRVRDYVVNGGRLIVTYYTGLSDEKDHVWLGGYPGSIRDVVGVRVEEFMPMGDDFPGVPDCLGLSNGAVAHDIADVIGSVDGTATVLETFRDDPWTGMDGAPAIVANTFGEGRSVYVGARLGRDGIAKSLPEIFESLGMAETGENDSRVLRVEREGSDGSRFVFSFNRTHEAVQIPFEGKIVVSSFAEVSGENVSIKPNGVIVTKQ

SEQ ID NO: 29 - protein, 1023 aa, Bifidobacterium catenulatum
  REGION 1..1023: note = >SEQ ID No: 29
MANSNRVEHASETWLTDATVFEVNRTPAHSNHKCFTHDPQSGEHSDLTQSLDGEWRVEIVQASDIDFNEEPFVAENFDDSSFCRAQVPGHLQMAGLLKNKYVNIQYPWDGHENPLEPNVPENNHVALYRRKFVVSKRLADTKESEGSVSIVFHGMATAIYVWVNGLFAGYGEDGFTPNEFDITDLLHDGENVVAVACYEYSSASWLEDQDFWRLHGLFRSVELTAQPHVHVENMQLEADWDAESGTASLDAALSVRNASDAATISATLKDSEGNVVWEASTNADANTTFASGSLQGLEPWSAESPSLYELEVNVIDQAGNIVEAAVQKVGFRRFRIENGIMTLNGKRIVFKGADRHEFDAKRGRSITEQDMIDDVIFCKRHNINAIRTSHYPNQERWYDLCDEYGIYLIDETNLETHGSWCLPGDVVTAETAVPGSKAHWEGACVDRVNSMVRRDYNHPSVVIWSLGNESYTGDVFRAMYKHVHDIDPNRPVHYEGVTKNRDYDDVTDIETRMYEHADVVEEYLKNDPQKPYISCEYMHAMGNSVGNLDEYTALERYPHYQGGFIWDFIDQAIYATQPDGSTRLCYGGDFGDRPSDYEFSGNGLVFADRTPTPKAQEVKQLYSNVHIDVTDRSVSIKNDNLFISTGGYQFVLRILADGEPVWQSERRFDVPADSACTFDVEWPVDLYRANADELVLEVSQRLAEATDWAPAGYELAFGQTIVAGTKAAEDAALPADGIVTVGRWNAGVQGSGREILLSRTQGGLVSYTFDGHEFVLRRPAITTFRALTDNDRGAGHGFERAQWMVAGRYARCVDNVIEQVDEDTLKAVYTYELATPQCTKVTVGYTADTTGRLNLHVEYPGESGELPTIPAFGIEWTLPVQYSNLRFFGAGPEETYQDRKHAKLGVWSTDAFKDHAPYLMPQETGNHEEVRWAEITDENGHGLRVSRANGAAPFAVSLQPYSSFMIEEAQHQDELPAPKHMFLRVLAAQMGVGGDDSWMSPVHSQYHITADQPISLDVNLELI

SEQ ID NO: 30 - protein, 1008 aa, Lactobacillus delbrueckii
  REGION 1..1008: note = >SEQ ID No: 30
  source 1..1008: note = subsp. bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 31 - protein, 1007 aa, Lactobacillus delbrueckii
  REGION 1..1007: note = >SEQ ID No: 31
  source 1..1007: note = subsp. lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLGFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLASLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 32 - protein, 1008 aa, Lactobacillus delbrueckii
  REGION 1..1008: note = >SEQ ID No: 32
  source 1..1008: note = subsp. bulgaricus
MSNKLVKEKRVDQADLAWLTDPEVYEVNTIPPHFDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 33 - protein, 1008 aa, Lactobacillus delbrueckii
  REGION 1..1008: note = >SEQ ID No: 33
  source 1..1008: note = subsp. bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGANRHEFDSKLGRAITEEDMIWDIKTMKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 34 - protein, 1008 aa, Lactobacillus delbrueckii
  REGION 1..1008: note = >SEQ ID No: 34
  source 1..1008: note = subsp. lactis
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNIQYPWDGSEEIFPPQVPSKNPLASYVRYFDLDEAFWDKEVSLKFAGAATAIYVWLNGHFVGYGEDSFTPSEFMVTKFLKKENNRLAVALYKYSSASWLEDQDFWRLSGLFRSVTLQAKPLLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTMKRSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGGDFDDRPTDYEFCGNGLVFADRTTSPKLANVKALYSNLKLEVKDGQLFLKNDNLFTNSSAYYFLTSLLVDGKLTYQSQPLTFGLEPGESGTFVLPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEAGNRSKVRYYQLFDEESGLEFTANGADLNLSALPYSAAQIEAADHAFELSNNYTWVRALAAQMGVGGDDSWGQKVHPEFCLDAQEARQLKLVIQPLLLK

SEQ ID NO: 35 - protein, 1008 aa, Lactobacillus delbrueckii
  REGION 1..1008: note = >SEQ ID No: 35
  source 1..1008: note = subsp. bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIRSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFDLTNNYTWVRALSAQMGVGGDDSWGQKVHPEFCLDAQKARQLRLVIQPLLLK

SEQ ID NO: 36 - protein, 1008 aa, Lactobacillus delbrueckii
  REGION 1..1008: note = >SEQ ID No: 36
  source 1..1008: note = subsp. bulgaricus
MSNKLVKEKRVDQADLAWLTDPEVYEVNTIPPHSDHESFQSQEELEEGKSSLVQSLDGDWLIDYAENGQGPVNFYAEDFDDSNFKSVKVPGNLELQGFGQPQYVNVQYPWDGSEEIFPPQIPSKNPLASYVRYFDLDEAFWDKEVSLKFDGAATAIYVWLNGHFVGYGEDSFTPSEFMVTKFLKKENNRLAVALYKYSSASWLEDQDFWRMSGLFRSVTLQAKPRLHLEDLKLTASLTDNYQKGKLEVEANIAYRLPNASFKLEVRDSEGDLVAEKLGPIGSEQLEFTLADLPVAAWSAEKPNLYQVRLYLYQAGSLLEVSRQEVGFRNFELKDGIMYLNGQRIVFKGVNRHEFDSKLGRAITEEDMIWDIKTIKRSNINAVRCSHYPNQSLFYRLCDKYGLYVIDEANLESHGTWEKVGGHEDPSFNVPGDDQHWLGASLSRVKNMMARDKNHASILIWSLGNESYAGTVFAQMADYVRKADPTRVQHYEGVTHNRKFDDATQIESRMYAPAKVIEEYLTNKPAKPFISVEYAHAMGNSVGDLAAYTALEKYPHYQGGFIWDWIDQGLEKDGHLLYGGDFDDRPTDYEFCGNGLVFADRTESPKLANVKALYANLKLEVKDGQLFLKNDNLFTNSSSYYFLTSLLVDGKLTYQSRPLTFGLEPGESGTFALPWPEVADEKGEVVYRVTAHLKEDLPWADEGFTVAEAEEVAQKLPEFKPEGRPDLVDSDYNLGLKGNNFQILFSKVKGWPVSLKYAGREYLKRLPEFTFWRALTDNDRGAGYGYDLARWENAGKYARLKDISCEVKEDSVLVKTAFTLPVALKGDLTVTYEVDGRGKIAVTADFPGAEEAGLLPAFGLNLALPKELTDYRYYGLGPNESYPDRLEGNYLGIYQGAVKKNFSPYLRPQETGNRSKVRWYQLFDEKGGLEFTANGADLNLSALPYSAAQIEAADHAFELTNNYTWVRALSAQMGVGGDDSWGQKVHPEFCLDAQKARQLRLVIQPLLLK

SEQ ID NO: 37 - protein, 1007 aa, Lactobacillus delbrueckii
  REGION 1..1007: note = >SEQ ID No: 37
  source 1..1007: note = subsp. lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKNPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTAFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 38 - protein, 628 aa, Lactobacillus helveticus
  REGION 1..628: note = >SEQ ID No: 38
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQDKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSDGGMGSYIKLLDKYPQYFGGFIWDFIDQALLVHDEISGHDVLRYGGDFDDRHSDYEFSGDGLMFADRTPKPAMQEVRYYYGLHK

SEQ ID NO: 39 - protein, 318 aa, Lactobacillus helveticus
  REGION 1..318: note = >SEQ ID No: 39
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDVGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPELDHEFSFILN

SEQ ID NO: 40 - protein, 1023 aa, Bifidobacterium longum
  REGION 1..1023: note = >SEQ ID No: 40
MTDVTHVDRASQAWLTDPTVFEVNRTPAHSSHKWYARDPQSGQWSDLKQSLDGEWRVEVVQAADINLEEEPATAESFDDSSFERIQVPGHLQTAGLMNHKYVNVQYPWDGHENPLEPNIPENNHVALYRRKFTVSAPVANAKQAGGSVSIVFHGMATAIYVWVNGAFVGYGEDGFTPNEFDITELLHDGENVVAVACYEYSSASWLEDQDFWRLHGLFRSVELAARPHVHIENTQIEADWDPEAGTASLDAALTVLNAADAATVRATLKDADGNTVWQTTGDAEAQTAISSGPLQGIAPWSAESPTLYELDVDVIDQAGDVIECTSQKVGFRRFRIEDGILTINGKRIVFKGADRHEFDAEQGRAITEQDMIDDVVFCKRHNINSIRTSHYPNQERWYELCDEYGIYLIDEANLEAHGSWSLPGDVLTEDTIVPGSKREWEGACVDRVNSMMRRDYNHPSVLIWSLGNESYVGDVFRAMYKHVHDIDPNRPVHYEGVTHNRDYDDVTDIETRMYSHADEIEKYLKDDPKKPYLSCEYMHAMGNSVGNMDEYTALERYPKYQGGFIWDFIDQAIYATQPDGTRSLRYGGDFGDRPSDYEFSGDGLLFADRKPSPKAQEVKQLYSNVHIDVTKDSVSVKNDNLFTATGDYVFVLSVLADGKPVWQSTRRFDVPAGETRTFDVAWPVAAYRADARELVLQVSQRLAKATDWAESGYELAFGQTVVPADATATPDTKPADGTITVGRWNAGVRGAGREVLLSRTQGGMVSYTFAGNEFVLRRPAITTFRPLTDNDRGAGHGFERVQWLGAGRYARCVDNVLEQIDDSTLKGTYTYELATAQRTKVTVSYTAHTDGRVNLHVEYPGEQGDLPTIPAFGIEWTLPVQYTNLRFFGTGPAETYLDRKHAKLGVWSTNAFADHAPYLMPQETGNHEDVRWAEITDDHGHGMRVSRADGAAPFAVSLLPYSSFMLEEAQHQDELPKPKHMFLRVLAAQMGVGGDDSWMSPVHPQYHIPADKPISLDVDLELI

SEQ ID NO: 41 - protein, 628 aa, Lactobacillus reuteri
  REGION 1..628: note = >SEQ ID No: 41
MDADIKWLDEPETFRVNQLPAHSDHYYYGNYDEWRHNNSRFAQNLDGQWQFNFAENLRERENDFYKMDYDSSSFGTIEVPSEIELNNYAQNNYINTLIPWEGKIYRRPAYTLSPDDAQEGSFSDGDDNTIGEYLKHFDLDPSLRGKQVRIRFDGVERAMYVWLNGHFIGYAEDSFTPSEFDLTPYIQDEGNVLAVEVFKHSTASWIEDQDMFRFSGIFRSVNLLAQPLVHVEDLNIRPIVTDNYQDGIFNVELQLHGEKTGNVNVRVIDNDGNTLVNETHPVDSTVKVQDQFLENVHLWDNHDPYLYQLLIEIRDDEGNLVELVPYRFGFRRIEINKDHVVLLNGQRLIINGVNRHEWDARRGRAITMDDMTSDIHTFKENNINAVRTCHYPDQIPWYYLCDDNGIYMMAENNLESHATWQKMGAIEPSYNVPGSVPQWRDVVVDRARTNYETFKNHPSILFWSLGNESYAGDNIVKMNEFYKKHDDSRLVHYEGVCHTPEYRDRISDVESWMYLPPKEVEEYLKNNPDKPFMECEYMHDMGNSDGGMGSYISLLDKYPQYFGGFIWDFIDQALLVKDPVSGQEVMRYGGDFDDRHSDYEFSGDGLMFADRTPKPAMQEVRYYYGLHK

SEQ ID NO: 42 - protein, 319 aa, Lactobacillus reuteri
  REGION 1..319: note = >SEQ ID No: 42
MAYTNKLRVIYGDATLGLSGDGFHYIFSYERGGLESLKLNGKEWLYREPMPTFWRATTDNDRGSGFNIRSAQWLAADTFHKCVGIDLTVDNQHFAELPIAPITNEFSDPVSAESVKIKYTFATLTVPATQVTVIYEVNGQGEIKVTMHYYGHEDLPGLPVVGMRFIMPTVATGFDYQGLSGETYPDRMAGATEGTFHVDGLPVTKYLVPQENGMHMATHALTITRDSTQNNADHSREPFSLTVKQDAQPFAFSCLPYTAEELENATHIEELPLARRTVLVVAGAVRGVGGIDSWGADVEEQYHIPADRDVEFSFVLNAK

SEQ ID NO: 43 - protein, 1007 aa, Lactobacillus delbrueckii
  REGION 1..1007: note = >SEQ ID No: 43
  source 1..1007: note = subsp. lactis
MSNKLVKEKRVDQADLAWLTDPEVYEVNTIPPHSDHESFQSQEELEEGKSSLVQSLDGNWLIDYAENGQGPINFYAEDFDDSNFKSVKVPGNLELQGFGQPQYVNIQYPWDGSEEIFPPQVPSKIPLASYVRYFDLDEALWDKEVSLKFAGAATAIYVWLNGHFVGYGEDSFTPSEFMVTKFLKKEGNRLAVALYKYSSASWLEDQDFWRLSGLFRSVTLEAKPLLHLEDLKLTASLTDNYQKGKLEVEANIAYRLPNASFKLEVRDSEGDLVAEKVGPIRSEKLDFSLADLPVAAWSAEKPNLYQVRLYLYQAGSLLEVSRQEVGFRNFELKDGIMYLNGQRIVFKGVNRHEFDSKLGRAITEADMIWDIKTMKQSNINAVRCSHYPNQSLFYRLCDKYGLYVIDEANLESHGTWEKVGHEDPSFNVPGDDQHWLGASLSRVKNMMARDKNHASILIWSLGNESYAGTVFAQMADYVRKADPTRVQHYEGVTHNRKFDDATQIESRMYAPAKEIEEYLTKKPAKPFISVEYAHAMGNSVGDLAAYTALEKYPHYQGGFIWDWIDQGLEKDGHLLYGGDFDDRPTDYEFCGDGLVFADRTTSPKLANVKALYSNLKLEVKDGQLFIKNDNLFTNSSAYYFLTSLLVDGKLTYQSQPLTFGLEPGESGTFALPWPEVEDEKGEIVYQVTAHLKEDLPWADEGFTVAEAEEAVTKLPEFYPAGRPELVDSDFNLGLKGNGFRILFSKAKGWPVSIKYAGREYLKRLPEFTFWRALTDNDRGAGYGYDLAKWENAGKYARLQDISYEIKENSALVKTTFTLPVALKGDLTITYEVDSLGKIAVTANFPGAVENGLLPAFGLNFALPKELSDYRYYGLGPNESYADRLEGSYLGIYQGMVEKNFTPYLRPQEAGNRSKVRYYQLFDEEGGLEFTANGADLNLSALPYSAAQIEAADHAFELTNNYTWVRALAAQMGVGGDDSWGQKVHPEFCLDAQEARQLKLVIQPLLLK

SEQ ID NO: 44 - protein, 628 aa, Lactobacillus helveticus
  REGION 1..628: note = >SEQ ID No: 44
MQANINWLDNPEVFRVNQLPAHSDHPFFRDYREWQKQHSSYQQSLNGKWKFHFSANPMDRPQDFYQRDFDSSNFDSIPVPSEIELSNYTQNQYINVLFPWEGKIFRRPAYALDPNDHEEGSFSKGADNTVGSYLKRFDLSSALIGKDVHIKFEGVEQAMYVWLNGHFVGYAEDSFTPSEFDLTPYIQEKDNLLAVEVFKHSTASWLEDQDMFRFSGIFRSVELLGIPATHLMDMDLKPRVADNYQDGIFNLKLHFIGKKAGSFHLLVKDIKGHTLLEKNEDIKENVQINNEKFENVHLWNNHDPYLYQLLIEVYDEQQNLLELIPFQFGFRRIEISPEKVVLLNGKRLIINGVNRHEWDAKRGRSITMSDMTTDINTFKENNINAVRTCHYPNQIPWYYLCDQNGIYVMAENNLESHGTWQKMGEIEPSDNVPGSIPQWKEAVIDRARNNYETFKNHTSILFWSLGNESYAGDNIIAMNEFYKSHDDTRLVHYEGVVHRPELKDKISDVESCMYLPPKKVEEYLQNDPPKPFMECEYMHDMGNSNGGMDSYIKLLDKYPQYFGGFIWDFIDQALLVHDEISGHDVLRYGGDFDDRHSDYEFSGDGLMFADRKPKPAMQEVRYYYGLHK

SEQ ID NO: 45 - protein, 318 aa, Lactobacillus helveticus
  REGION 1..318: note = >SEQ ID No: 45
MDYTNNQLHIIYGDATFGVNGKDFQYIFSYERGGLESLKVHGKEWLYRVPTPTFWRATTDNDRGSGFNLKAAQWLGADMFTKCTDIHLKVDRHDFAELPIAPFNNKFSNHEYAKSAEISFTYQTLTTPATNAKIIYNIDDGGHIKVTMRYYGKKGLPPLPVIGIRLIMPTAATGFDYEGLSGETYPDRMAGAKEGKFHIDGLPVTEYLVPQENGMHMQTKKLTINRETTQNNVDRTNEKFSLSIQQAEKPFNFSCLPYTAEELENATHIEELPLVRRTVLVIAGAVRGVGGIDSWGTDVESAYHINPDLDHEFSFILN

SEQ ID NO: 46 - protein, 626 aa, Lactobacillus crispatus
  REGION 1..626: note = >SEQ ID No: 46
MKANIKWLDDPEVFRINQLPAHSDHPFYKDYREWQKHSSSFKQSLNGAWQFHFSKDPQSRPIDFYKLSFDSSSFDTIPVPSEIELNGYAQNQYTNILYPWESKIYRKPAYTLGRGIKDGDFSQGKDNTVGSYLKHFDLNPALAGHDIHIQFEGVERAMYVYLNGHFIGYAEDSFTPSEFDLTPYIQAKDNILAVEVFKHSTASWLEDQDMFRFSGIFRSVELLALPRTHLMDLDIKPTVVNDYHDGVFNAKLHFMGKTSGNVHVLIEDIDGKTLLNKKLPLKSTVEIENETFANVHLWDNHDPYLYQLIIEVHDQDGKLVELIPYQFGFRKIEITKDHVVLLNGKRLIINGVNRHEWDAKRGRSITLADMKQDIATFKHNNINAVRTCHYPNQIPWYYLCDQNGIYMMAENNLESHGTWQKLGQVEATSNVPGSIPEWREVVVDRARSNYETFKNHTAILFWSLGNESYAGSNIAAMNKLYKDHDSSRLTHYEGVFHAPEFKKEISDLESCMYLPPKEAEEYLQNPKKPLVECEYMHDMGNSDGGIGSYIKLIDKYPQYMGGFIWDFIDQALLVHDPVSGQDVLRYGGDFDDRHSDYEFSGDGLMFADRTPKPAMQEVRYYYGLHK

SEQ ID NO: 47 - protein, 316 aa, Lactobacillus crispatus
  REGION 1..316: note = >SEQ ID No: 47
MAYTNNLHVVYGEASLGVNGQDFAYLFSYERGVLESLKIKDKEWLYRTPTPTFWRATTDNDRGSGFNQKAAQWLGADMFTKCVGIHVQVDDHQFDELPIAPINNQFSNQEFAHEVKVAFDYETLTTPATKVKIIYNINDAGHMTITMHYFGKKGLPPLPVIGMRFIMPTKAKSFDYTGLSGETYPDRMAGAERGTFHIDGLPVTKYLVPQENGMHMQTNELVITRNSTQNNADKDGDFSLKITQTKQPFNFSLLPYTAEELENATHIEELPLARRSVLVIAGAVRGVGGIDSWGSDVEEQYHIDPEQDHEFSFTLN

SEQ ID NO: 48 - protein, 1025 aa, Streptococcus thermophilus
  REGION 1..1025: note = >SEQ ID No: 48
MNMTKIQTYLNDPKIVSVNTVDAHSDHKYFESLEEFSEGEMKLRQSLNGKWKIHYAQNTNQVLKDFYKTEFDETDLNFINVPGHLELQGFGSPQYVNTQYPWDGKEFLRPPQVPQESNAVASYVKHFTLNDALKDKKVFISFQGVATSIFVWVNGNFVGYSEDSFTPSEFEISDYLVEGDNKLAVAVYRYSTASWLEDQDFWRLYGIFRDVYLYAIPKVHVQDLFVKGDYDYQTKAGQLDIDLKTVGDYEDKKIKYVLSDYEGIVTEGDASVNGDGELSVSLENLKIKPWSAESPKLYDLILHVLDDDQVVEVVPVKVGFRRFEIKDKLMLLNGKRIVFKGVNRHEFNARTGRCITEEDMLWDIKVMKQHNINAVRTSHYPNQTRWYELCDEYGLYVIDEANLETHGTWQKLGLCEPSWNIPASEPEWLPACLDRANNMFQRDKNHASVIIWSCGNESYAGKDIADMADYFRSVDNTRPVHYEGVAWCREFDYITDIESRMYAKPADIEEYLTTGKLVDLSSVSDKHFASGNLTNKPQKPYISCEYMHTMGNSGGGLQLYTDLEKYPEYQGGFIWDFIDQAIYKTLPNGSEFLSYGGDWHDRPSDYEFCGNGIVFADRTLTPKLQTVKHLYSNIKIAVDEKSVTIKNDNLFEDLSAYTFLARVYEDGRKVSESEYHFDVKPGEEATFPVNFVVEASNSEQIYEVACVLREATKWAPKGHEIVRGQYVVEKISTETPVKAPLNVVEGDFNIGIQGQNFSILLSRAQNTLVSAKYNGVEFIEKGPKLSFTRAYTDNDRGAGYPFEMAGWKVAGNYSKVTDTQIQIEDDSVKVTYVHELPGLSDVEVKVTYQVDYKGRIFVTANYDGKAGLPNFPEFGLEFAIGSQFTNLSYYGYGAEESYRDKLPGAYLGRYETSVEKTFAPYLMPQESGNHYGTREFTVSDDNHNGLKFTALNKAFEFSALRNSTEQIENARHQYELQESDATWIKVLAAQMGVGGDDSWGAPVHDEFLLSSADSYQLSFMIEPLN

SEQ ID NO: 49 - protein, 1008 aa, Lactobacillus delbrueckii
  REGION 1..1008: note = >SEQ ID No: 49
  source 1..1008: note = subsp. indicus
MNNKLAQVKRVDQADLAWLTDPEIYEVNTIPPHSDHESFQSLEELEEGKSSLVQSLDGDWLIDYAENGEGPANFYEENFDDSSFKSVKVPGNLELQGFGQPQYVNVQYPWDGSDEIFPPMIPSKNPVASYVRYFDLEEAFWDKEVSLKFAGAATAIYVWLNGHFVGYGEDSFTPSEFMVTKFLKKEGNRLAVALYKYSSASWLEDQDFWRMSGLFRSVTLEAKPLLHLQDLKLTASLTNDYQKGSLQVEADIDYRLPNSSFKLELRDSAGELVAEKVGPIRSEKLDFSLADLPVAAWSAEEPNLYQVRLSLYQQGSLLEVSRQEVGFRNFELKDGIMYLNGKRIVFKGVNRHEFDSKLGRAITEADMIWDIKTMKQSNINAVRCSHYPNQSIFYHLCDKYGLYVIDEANLESHGTWEKVGGHEDPSFNVPGDDQRWLGASLSRVKNMMARDKNHASILIWSLGNESYAGKVFAQMADYVRQADPTRVQHYEGVTHNRKFDDATQIESRMYAPAKEIEEYLTKKPAKPFVSCEYAHAMGNSVGDLAAYTALEKYPHYQGGFIWDWIDQGLEKEGHLLYGGDFDDRPSDYEFCGDGLVFADRTTSPKLANVKALYSNLKLELKDGQLFLKNDNLFTNSSAYYFLTSLLVDGKLTYQSQPLTFALEPGESGTFALPWPEVEDEKGEIVYQVTAHLKEDLPWADEGFTVAEAEEAVTKLPEFYPAGRPELVDSDYNLGIKGNGFRILFSKAKGWPVSIKYAGREYLKRLPEFTFWRALTDNDRGAGYGYDLAKWENAGKYARLQDISYEIKENSVLVKTAFTLPVALKGDLTITYEVDSLGKIAVTANFPGAVENGLLPAFGLNFALPKELSDYRYYGLGPNESYADRLEGSYLGIYQGAVEKNFTPYLRPQEVGNRSKVRYYQLFDEEGGLEFTANGANLNLSALPYSAAQIEAADHAFELTNNYTWVRALAAQMGVGGDDSWGQKVHPEFCLDAQEARQLKLVIQPLFTE

SEQ ID NO: 50 - protein, 1049 aa, Bifidobacterium adolescentis
  REGION 1..1049: note = >SEQ ID NO: 50
MADTAELAIVHATTASASWLTDPTVFAANRKPAHSSHRYVIGETSEPKQSLDGEWKVRIEQARNVDVESAPFAAVDFEDGDFGAIEVPGHLQMAGYLKNKYVNIQYPWDGHEDPQAPNIPENNHVAIYRRRFALDAQLARTLENDGTVSLTFHGAATAIYVWLDGTFVGYGEDGFTPSEFDVTEALRNGNGNAADSPEAEHTLTVACYEYSSASWLEDQDFWRLHGLFRTVELAAQPHTHVETVQLEADYTAADTAGTADTAELNAALTLRNPADAMTIESTLRDGDGNVVWESTQACNGEIALNSGKMTNIAPWSAESPTLYTLTVRVVGHDGAIIETVTQKIGFRTFRIENGIMTLNGKRIVFKGADRHEFDAKRGRAITREDMLSDVVFCKRHNINAIRTSHYPNQEYWYDLCDEYGLYLIDETNMETHGTWVANNVERPEDGIPGSRPEWEDACVDRINSMMRRDYNHPSVLIWSLGNESSAGEVFRAMYRHAHTIDPNRPVHYEGSVHMREFEDVTDIESRMYAHADEIERYLNDGSPAHTDGPKKPYISCEYMHAMGNSCGNMDEYTALERYPMYQGGFIWDFIDQAIETKLPDGTTRMCYGGDFGDRPSDYEFSGDGLLFADRTPSPKAQEVKQLYANVKIAVSVDEARITNDNLFVSTGDYRFVLRILADGKPVWSTTRRFDVAAGESASFEVDWPVDDYRSNAEELVLEVSQQLGNACDWAPAGYELAFGQCVVAGAKTTADAVDAAGAPADGTVTLGRWNAGVRGQGREALFSRTQGGMVSYTFGEREFVLRRPSITTFRPLTDNDRGAGHAFERAAWAVAGKYARCVDCAIANRGENAVEATYTYELAIPQRTKVTVRYVADTAGLVSLDVEYPGEKNGDLPTIPAFGIEWALPVEYANLRFYGAGPEETYADRRHAKLGVWSTTAGDDCAPYLLPQETGNHEDVRWAEITDDSGHGVRVKRGAGAKPFAMSLLPYSSTMLEEALHQDELPKPRHMFLRLLAAQMGVGGDDSWMSPVHEQYQLPADQPLSLNVQLKLF

SEQ ID NO: 51 - protein, 1023 aa, Bifidobacterium adolescentis
  REGION 1..1023: note = >SEQ ID NO: 51
MANETRIEHASETWLADSTVFEVNRVPAHSDHKCYAHDSQTNEWSDLRQSLDGEWRVEVVQASDIEFNEEPFVRENFDDSAFERIQVPGHLQMAGLMNNKYVNIQYPWDGHENPAEPNIPENNHVALYRKTFTMANRLADTKNAGGTVSIVFHGMATAIYVWVNGMFVGYGEDGFTPNEFDITEMLHDGENVVAVACYEYSSASWLEDQDFWRLHGLFRSVELAAQPHVHIENMQIESDWDPESGSASLDAALTVRNAADAATISATLKDSDGNVVWETANCADPDTSISTGSLNGIRPWSAEDPVLYEFEVTVIDHAGNIAEVAVQKVGFRRFRIEDGIMTINGKRIVFKGADRHEFDPKRGRAITEQDMIDDVVFCKRHNLNAIRTSHYPNQERWYELCDEYGIYLIDETNLETHGSWCLPGDVLTEETAVPGSKAHWEGACVDRVNSMVRRDYNHPSVLIWSLGNESYTGDVFRAMYKRVHDIDPNRPVHYEGVTHNRDYNDVTDIETRMYAHADAIEEYLKNDPQKPYISCEYMHAMGNSCGNMDEYTALERYPKYQGGFIWDFIDQAIYATQPDGTTSLRYGGDFGDRPSDYEFSGNGLVFADRKPTPKAQEVKQLYSNVHIDVAEDSVTIKNDNLFTSTGEYTFVLRVLADGEPVWQSERRFDVPAGSTEKLDVDWPLDLYRDGASELVLEVSQRLAKATNWAVAGYELAFGQTVVAGSKKASAPVKPVDGIVTVGRWNVGVQGSGREVLLSRTQGGLVSYTFNNREFVLRRPAVTTFRALTDNDRGAGHGFERAQWLGAGRYARCIGNEIEQIDENTVKASYTYELATPQRTKVTVSYTADTTGRVNLHVEYPGEPGDLPTIPAFGIEWTLPVQYSNLRFFGAGPEETYQDRKHAKLGVWSTDAFKDHAPYLMPQETGNHEDVRWAEITDEKGHGLRISRAEGAEPFAMSLQPYSSFMLEEAQHQDELPAPKHMFLRVLAEQMGVGGDDSWMSPVHPQYHIPADQPISLDVDLDLI

SEQ ID NO: 52 - protein, 1305 aa, Bifidobacterium bifidum
  REGION 1..1305: note = >SEQ ID NO: 52
MVEDATRSDSTTQMSSTPEVVYSSAVDSKQNRTSDFDANWKFMLSDSVQAQDPAFDDSAWQQVDLPHDYSITQKYSQSNEAESAYLPGGTGWYRKSFTIDRDLAGKRIAINFDGVYMNATVWFNGVKLGTHPYGYSPFSFDLTGNAKFGGENTIVVKVENRLPSSRWYSGSGIYRDVTLTVTDGVHVGNNGVAIKTPSLATQNGGNVTMNLTTKVANDTEAAANITLKQTVFPKGGKTDAAIGTVTTASKSIAAGASADVTSTITAASPKLWSIKNPNLYTVRTEVLNGDTVLDTYDTEYGFRWTGFDATSGFSLNGEKVKLKGVSMHHDQGSLGAVANRRAIERQVEILQKMGVNSIRTTHNPAAKALIDVCNEKGVLVVEEVFDMWNRSKNGNTEDYGKWFGQTIAGDNAVLGGDKDETWAKFDLTSTINRDRNAPSVIMWSLGNEMMEGISGSVSDFPATSAKLVAWTKAADSTRPMTYGDNKIKANWNESNTMGDNLTANGGVVGTNYSDGANYDKIRTTHPSWAIYGSETASAINSRGIYNRTTGGAQSSDKQLTSYDNSAVGWGAVASSAWYDVVQRDFVAGTYVWTGFDYLGEPTPWNGTGSGAVGSWPSPKNSYFGIVDTAGFPKDTYYFYQSQWNDDVHTLHILPAWNENVVAKGSGNKVPVVVYTDAAKVKLYFTPKGSTEKRLIGEKSFTKKTTAAGYTYQVYEGTDKDSTAHKNMYLTWNVPWAEGTISAEAYDENNRLIPEGSTEGNASVTTTGKAAKLKADADRKTITADGKDLSYIEVDVTDANGHIVPDAANRVTFDVKGAGKLVGVDNGSSPDHDSYQADNRKAFSGKVLAIVQSTKEAGEITVTAKADGLQSSTVKIATTAVPGTSTEKTVRSFYYSRNYYVKTGNKPILPSDVEVRYSDGTSDRQNVTWDAVSDDQIAKAGSFSVAGTVAGQKISVRVTMIDEIGALLNYSASTPVGTPAVLPGSRPAVLPDGTVTSANFAVHWTKPADTVYNTAGTVKVPGTATVFGKEFKVTATIRVQRSQVTIGSSVSGNALRLTQNIPADKQSDTLDAIKDGSTTVDANTGGGANPSAWTNWAYSKAGHNTAEITFEYATEQQLGQIVMYFFRDSNAVRFPDAGKTKIQISADGKNWTDLAATETIAAQESSDRVKPYTYDFAPVGATFVKVTVTNADTTTPSGVVCAGLTEIELKTATSKFVTNTSAALSSLTVNGTKVSDSVLAAGSYNTPAIIADVKAEGEGNASVTVLPAHDNVIRVITESEDHVTRKTFTINLGTEQEFPADSDERD

SEQ ID NO: 53 - protein, 1025 aa, Kluyveromyces lactis
  REGION 1..1025: note = >SEQ ID No: 53
MSCLIPENLRNPKKVHENRLPTRAYYYDQDIFESLNGPWAFALFDAPLDAPDAKNLDWETAKKWSTISVPSHWELQEDWKYGKPIYTNVQYPIPIDIPNPPTVNPTGVYARTFELDSKSIESFEHRLRFEGVDNCYELYVNGQYVGFNKGSRNGAEFDIQKYVSEGENLVVVKVFKWSDSTYIEDQDQWWLSGIYRDVSLLKLPKKAHIEDVRVTTTFVDSQYQDAELSVKVDVQGSSYDHINFTLYEPEDGSKVYDASSLLNEENGNTTFSTKEFISFSTKKNEETAFKINVKAPEHWTAENPTLYKYQLDLIGSDGSVIQSIKHHVGFRQVELKDGNITVNGKDILFRGVNRHDHHPRFGRAVPLDFVVRDLILMKKFNINAVRNSHYPNHPKVYDLFDKLGFWVIDEADLETHGVQEPFNRHTNLEAEYPDTKNKLYDVNAHYLSDNPEYEVAYLDRASQLVLRDVNHPSIIIWSLGNEACYGRNHKAMYKLIKQLDPTRLVHYEGDLNALSADIFSFMYPTFEIMERWRKNHTDENGKFEKPLILCEYGHAMGNGPGSLKEYQELFYKEKFYQGGFIWEWANHGIEFEDVSTADGKLHKAYAYGGDFKEEVHDGVFIMDGLCNSEHNPTPGLVEYKKVIEPVHIKIAHGSVTITNKHDFITTDHLLFIDKDTGKTIDVPSLKPEESVTIPSDTTYVVAVLKDDAGVLKAGHEIAWGQAELPLKVPDFVTETAEKAAKINDGKRYVSVESSGLHFILDKLLGKIESLKVKGKEISSKFEGSSITFWRPPTNNDEPRDFKNWKKYNIDLMKQNIHGVSVEKGSNGSLAVVTVNSRISPVVFYYGFETVQKYTIFANKINLNTSMKLTGEYQPPDFPRVGYEFWLGDSYESFEWLGRGPGESYPDKKESQRFGLYDSKDVEEFVYDYPQENGNHTDTHFLNIKFEGAGKLSIFQKEKPFNFKISDEYGVDEAAHACDVKRYGRHYLRLDHAIHGVGSEACGPAVLDQYRLKAQDFNFEFDLAFE

(19) United States
(12) Patent Application Publication
(10) Pub. No.: US 2026/0033510 A1
(43) Pub. Date: Feb. 5, 2026

(54) IMPROVING FILTERABILITY OF LACTASE BY ADDING ANIONS SELECTED FROM MALATE, TARTRATE, CITRATE, GLUCONATE, EDTA OR COMBINATIONS THEREOF AND STERILE FILTERED LACTASE PRODUCT OBTAINED

(71) Applicant: Kerry Group Services International Ltd, Co. Kerry (IE)

(72) Inventor: Miguel Toscano, Hoersholm (DK)

(21) Appl. No.: 18/872,758

(22) PCT Filed: May 26, 2023
(86) PCT No.: PCT/EP2023/064224

§ 371 Date: Dec. 6, 2024

(30) Foreign Application Priority Data
Jun. 7, 2022 (EP) 22177449.0

(51) Int. Cl. A23C 9/12; A23C 9/13; C12N 9/24

(52) U.S. Cl. CPC A23C 9/1206; A23C 9/1307; C12N 9/2402; C12Y 302/01108

(57) ABSTRACT

Sterile filtered liquid lactase composition comprising a lactase, preferably a neutral or acidic lactase, an anion selected from malate, tartrate, citrate, gluconate and/or EDTA and a cation selected from sodium and potassium. Preferably, the composition further comprises a polyol. Addition of the said anions to lactase improves filterability by reducing pressure in a filtration system. Preferably the filtration forms part of an in-line filtration system in the production of a dairy product.

### CROSS-REFERENCE TO RELATED APPLICATIONS

This application claims the benefit of priority to international patent application number PCT/EP2023/064224, filed May 26, 2023, which claims the benefit of priority to European patent application number 22177449, filed Jun. 7, 2022, each of which is hereby expressly incorporated by reference in its entirety.

### REFERENCE TO SEQUENCE LISTING

The present application is being filed along with a Sequence Listing in electronic format. The Sequence Listing is provided as a file entitled BRDIT93.009APC_REPLACEMENT_SEQ_LIST.xml, which was created on Nov. 15, 2024, was last modified Oct. 15, 2025, and which is 120,831 bytes in size. The information in the electronic Sequence Listing is hereby incorporated by reference in its entirety.

### TECHNICAL FIELD

The present invention generally relates to an improved formulation or improved composition of a lactase or lactase product, wherein said formulation or composition is supplemented with an anion selected from malate, tartrate, citrate, gluconate, ethylenediaminetetraacetate, or combinations thereof. This formulation or composition contributes to the improvement of the physical stability and filterability of a lactase or lactase product.

### BACKGROUND

Currently, lactases or lactase products are formulated with glycerol and tap water only. The addition of a lactase or lactase product to milk or to a milk-based product may be carried out before or after the sterilization of milk or milk-based product. In the case of the latter, a step for sterilizing the lactase by filtration is needed. However, a lactase or lactase product can easily cause filter clogging during the step of sterilizing the lactase by filtration. This problem has been reported in several documents, such as EP 3 568 023 or EP 3 187 582, and is responsible for the replacement of multiple filters, leading to an increase in production costs and a delay in production.

### SUMMARY

The objective of the present invention or disclosure is to provide a formulation or composition of a lactase or lactase product which avoids blockage of a filter, or in other words a formulation or a composition of a lactase or lactase product having an improved physical stability and/or filterability. This objective is achieved when said formulation or composition is supplemented by an anion selected from a list consisting of malate, tartrate, citrate, gluconate, ethylenediaminetetraacetate, and combinations thereof, leading to the improvement of the physical stability and/or filterability of the formulation or composition of the lactase or lactase product. In the context of the present invention or disclosure, the improvement of the physical stability and/or filterability is observed by reducing the pressure measured in the filtering system when a formulation or composition comprising a lactase or lactase product and an anion such as malate, tartrate, citrate, gluconate, ethylenediaminetetraacetate, and/or combinations thereof is filtered versus the control. In the context of the present invention or disclosure, the control is defined as a formulation or composition comprising a lactase or lactase product but deprived of a supplemented anion selected from malate, tartrate, citrate, gluconate, ethylenediaminetetraacetate, and/or combinations thereof.

The present invention or disclosure shows that an anion selected from malate, tartrate, citrate, gluconate, and/or combinations thereof contributes to the reduction of the pressure buildup in the filtration system and, thereby, allows longer filtration times before reaching to the pressure limits of the system, therefore preventing the disruption of the filter. As a result, higher volumes of a formulation or composition comprising a lactase or lactase product and said anion can be filtered using the same filter in comparison with the volumes that can be filtered when a formulation or composition lactase or lactase product or lactase deprived of said anion is filtered before the filter needs to be replaced.

None of the above mentioned documents refer, hint or indicate that the nature of the anion would influence filterability of a composition comprising a lactase and said anion.

### Definitions

In connection with the present invention or disclosure the following definitions apply.

“Neutral lactase” as used herein refers to a lactase which is enzymatically active, preferably has an optimal activity, at a neutral pH.

“Acidic lactase” as used herein refers to a lactase which is enzymatically active, preferably has an optimal activity, at an acidic pH.

“Sequence identity” for amino acids as used herein refers to the sequence identity calculated as (n_{ref} n_{dif})·100/n_{ref}, wherein n_{dif} is the total number of non-identical residues in the two sequences when aligned and wherein n_{ref} is the number of residues in one of the sequences. In some embodiments the sequence identity is determined by conventional methods, e.g., Smith and Waterman, 1981, Adv. Appl. Math. 2:482, by the search for similarity method of Pearson & Lipman, 1988, Proc. Natl. Acad. Sci. USA 85:2444, using the CLUSTAL W algorithm of Thompson et al., 1994, Nucleic Acids Res 22:467380, by computerized implementations of these algorithms (GAP, BESTFIT, FASTA, and TFASTA in the Wisconsin Genetics Software Package, Genetics Computer Group). The BLAST algorithm (Altschul et al., 1990, Mol. Biol. 215:403-10) for which software may be obtained through the National Center for Biotechnology Information www.ncbi.nlm.nih.gov/) may also be used. When using any of the aforementioned algorithms, the default parameters for “Window” length, gap penalty, etc., are used.

Composition and formulation are herein synonymously used.

Sodium citrate means trisodium citrate.

The use of the terms “a” and “an” and “the” and similar referents in the context of describing the invention or disclosure (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention or disclosure and does not pose a limitation on the scope of the invention or disclosure unless otherwise claimed. No language in the specification should be construed as indicating any non-claimed element as essential to the practice of the invention or disclosure.

### DETAILED DESCRIPTION

This invention or disclosure relates a composition comprising a lactase and an anion selected from malate, tartrate, citrate, gluconate, ethylenediaminetetraacetate, and/or a combination thereof. The counterion (or cation in this case) may be selected from sodium and potassium, as seen from the examples. Preferably, the composition herein disclosed may comprise 0.1-250 mM of malate, 0.1-250 mM of tartrate, 0.1-250 mM of citrate, 0.1-250 mM of gluconate, 0.1-250 mM of ethylenediaminetetraacetate, and/or a combination thereof. More preferably, said composition may comprise 1-150 mM of malate, 1-150 mM of tartrate, 1-150 mM of citrate, 1-150 mM of gluconate, 1-150 mM of ethylenediaminetetraacetate, and/or a combination thereof. Even more preferably, said composition may comprise 2-100 mM or 10-20 mM of malate, 2-100 mM or 10-20 mM of tartrate, 2-100 mM or 10-20 mM of citrate, 2-100 mM or 10-20 mM of gluconate, 2-100 mM or 10-20 mM of ethylenediaminetetraacetate, and/or a combination thereof.

This invention or disclosure also relates to a composition comprising a lactase and a salt selected from a list consisting of sodium malate, potassium malate, sodium tartrate, potassium tartrate, sodium citrate, potassium citrate, sodium gluconate, potassium gluconate, ethylenediaminetetraacetate disodium salt, ethylenediaminetetraacetate dipotassium salt, and/or a combination thereof. Preferably, the composition herein disclosed may comprise 0.1-250 mM of sodium malate, potassium malate, sodium tartrate, potassium tartrate, sodium citrate, potassium citrate, sodium gluconate, potassium gluconate, ethylenediaminetetraacetate disodium salt, ethylenediaminetetraacetate dipotassium salt, and/or a combination thereof. More preferably, said composition may comprise 1-150 mM of sodium malate, potassium malate, sodium tartrate, potassium tartrate, sodium citrate, potassium citrate, sodium gluconate, potassium gluconate, ethylenediaminetetraacetate disodium salt, ethylenediaminetetraacetate dipotassium salt, and/or a combination thereof. Even more preferably, said composition may comprise 2-100 mM or 10-20 mM of sodium malate, potassium malate, sodium tartrate, potassium tartrate, sodium citrate, potassium citrate, sodium gluconate, potassium gluconate, ethylenediaminetetraacetate disodium salt, ethylenediaminetetraacetate dipotassium salt, and/or a combination thereof.

Preferably, any of the compositions herein disclosed may comprise 20 and 10000 BLU/g of a lactase.

Preferably, any of the compositions herein disclosed may comprise a neutral lactase or an acidic lactase.

Preferably, said lactase may have an amino acid sequence which is at least least 75%, or at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53. The most preferred sequences are SEQ ID NO: 1, 7, 40, 52, 53 and any sequence having 75%-100% or 80%-100% or 85%-100% identical to any of SEQ ID NO: 1, 7, 40, 52, 53, such as 86%, 87%, 88%, 89%, 90%, 91%, 92%, 93%, 94%, 95%, 96%, 97%, 98%, 99%, or 100% identical to any of SEQ ID NO: 1, 7, 40, 52, 53.

Any of the compositions herein disclosed may further comprise a polyol selected among glycerol, sorbitol, monopropylene glycol, sucrose, glucose, galactose or lactose, or a combination thereof. Preferably, said polyol may be present at a concentration of 10-70% w/w, more preferably 20-80% w/w, even more preferably 30-60% w/w. Preferably, the polyol is or may be glycerol.

Additionally, any of the compositions herein disclosed may be liquid lactase composition, preferably a sterile liquid lactase composition or a sterile-filtered liquid lactase composition. The present invention or disclosure also concerns the use of an anion selected from malate, tartrate, citrate, gluconate, ethylenediaminetetraacetate, and/or a combination thereof, for improving the filterability of any of the compositions herein disclosed. The counterion (or cation in this case) may be sodium and/or potassium, as seen from the examples.

The present invention or disclosure also concerns the use of an anion selected from malate, tartrate, citrate, gluconate, ethylenediaminetetraacetate, and/or a combination thereof, reducing pressure of a filtration system, preferably for reducing pressure of an in-line filtration system of a production process of a dairy product.

Preferably, the use herein disclosed relates to 0.1-250 mM of malate, 0.1-250 mM of tartrate, 0.1-250 mM of citrate, 0.1-250 mM of gluconate, 0.1-250 mM of ethylenediaminetetraacetate, and/or a combination thereof. More preferably, it relates to 1-150 mM of malate, 1-150 mM of tartrate, 1-150 mM of citrate, 1-150 mM of gluconate, 1-150 mM of ethylenediaminetetraacetate, and/or a combination thereof. Even more preferably, it relates to 2-100 mM or 10-20 mM of malate, 2-100 mM or 10-20 mM of tartrate, 2-100 mM or 10-20 mM of citrate, 2-100 mM or 10-20 mM of gluconate, 2-100 mM or 10-20 mM of ethylenediaminetetraacetate, and/or a combination thereof.

The present invention or disclosure also relates to a method for reducing pressure in a filtration system comprising the step of filtrating any of the lactase compositions herein disclosed. Preferably wherein the filtration system is a in-line filtration system of a production process of a dairy product. More preferably, wherein the filtration system is an aseptic in-line dosing system.

### EXAMPLES

Any lactase or lactase product, in particular showing poor filterability, can be used in the examples below. For example, the lactase or lactase product may comprise a lactase from Bifidobacterium, Lactobacillus, Streptococcus, or Kluyveromyces. Preferably, the lactase or lactase product may comprise a lactase from Bifidobacterium adolescentis, Bifidobacterium bifidum, Bifidobacterium breve, Bifidobacterium catenulatum, Bifidobacterium longum, Lactobacillus sakei, Lactobacillus amylovorus, Lactobacillus delbrueckii such as Lactobacillus delbrueckii subsp. lactis or Lactobacillus delbrueckii subsp. bulgaricus or Lactobacillus delbrueckii subsp. indicus, Lactobacillus helvaticus, Lactobacillus reuteri, Lactobacillus crispatus, Streptococcus thermophilus, Kluyveromyces lactis.

More preferably, the lactase or lactase product represented by any of the sequences herein disclosed as SEQ ID NO: 1 to 53 may be used in the present invention or disclosure. A lactase or lactase product having an amino acid sequence which is at least 75%, or at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53 may be used in the present invention or disclosure. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53 may be used in the present invention or disclosure. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53 may be used in the present invention or disclosure. The combination of a lactase or a lactase product with an anion such as malate, tartrate, citrate, gluconate, ethylenediaminetetraacetate, and/or combinations thereof results in a formulation or composition comprising said lactase or lactase product and said anion. The counterion (or cation in this case) may be sodium or potassium, as seen from the examples.

An example of a Bifidobacterium bifidum lactase is NOLA® Fit from Chr. Hansen A/S, Saphera® from Novozymes A/S or Nurica™ from IFF. An example of a Kluyveromyces lactis lactase is Ha-Lactase™ from Chr. Hansen A/S, Lactozym® from Novozymes A/S, Maxilact® from DSM.

An example of a Lactobacillus delbrueckii subsp. bulgaricus is Bonlacta™ from IFF.

In the examples below, the pressure remaining in the system when the control formulation or composition is filtered is measured and normalized to 100%. Within the same example, the pressure remaining in the system when a formulation or composition comprising a lactase or lactase product and an anion selected from malate, tartrate, citrate, gluconate, and/or combinations thereof (also named as tested formulation or tested composition) was measured and normalized in view of the control formulation used in the same example. For the sake of completeness, same volumes of the control formulation (or control composition) and of the tested formulations (or tested compositions) were filtered. Additionally, for each sample 2 independent measurements were made.

### Example 1

NOLA© Fit 5500 batches (from Chr. Hansen A/S) were diluted to a 50% (v/v) glycerol in tap water (control) or were diluted to a 50% (v/v) glycerol in tap water supplemented with an anion selected from malate, tartrate, citrate, gluconate, ethylenediaminetetraacetate, or a combination thereof, such that a final strength of 2750 BLU/g strength was reached.

The resulting composition were pre-filtered through a 0.45 μm VWR Media Bottle top filtration unit, PES 0.45 μm, 90 mm (Cat no #514-0300), followed by storage at 5° C. After incubation at 37° C. for 24 h, the samples are brought to room temperature for 1 h before the filterability test. Preferably the samples were brought to a temperature ranging from 15 to 30° C., preferably 15 to 25° C., more preferably 20 to 25° C. (at 1 atm). The samples are filtered using a SartoFLOW Smart filtration unit, through a 0.45 μm pre-filter (Sartorius Syringe Filter, Minisart NML, CA, 28 mm, 0.45 μm, sterile, Cat #16555-K) and a 0.2 μm sterile filter (Pall Life Science Acrodisc 25 mm Syringe Filter 0.2 μm Fluorodyne II membrane (sterile) VWR cat #516-8973P) in sequence and pre-wetted with MilliQ Water (ELGA Purelab flex). The flow rate was kept stable at 6.5 g/min (+/−10%), and the initial rate of increase in back pressure on the system was measured and calculated in bar/hour, through a linear fit.

The increase in the pressure rate of the system was used to evaluate filterability of the samples, meaning that samples that result in higher pressure increase rates have lower filterability.

TABLE 1
Sample 1 submitted to a stress period of 37° C. for 24 h.

| | | | Pressure remaining | Pressure |
|---|---|---|---|---|
| | | | in the system | reduced by the |
| Sample | | | vs control in % | composition in % |
| 1 | Control | 0 mM | 100 | — |
| | NaCl | 200 mM | 92 | 8 |
| | Na₃citrate | 10 mM | 41 | 59 |

Table 1 shows that citrate, in particular sodium citrate, performs better than the control or better than chloride, in particular sodium chloride, in reducing the pressure generated when filtering a lactase composition. As a result, a higher volume of a lactase composition comprising citrate can be filtered before the filter is clotted versus the volume of a control lactase composition or, in alternative, comprising sodium chloride.

Additionally, Table 1 shows that a lower concentration of citrate, in particular sodium citrate, is needed to promote a reduction of the pressure of the filtration system in contrast to the one needed when chloride or sodium chloride is used. For example, 10 mM of sodium citrate (about 0.3% w_{sodium citrate}/w_{composition of lactase}) contributes to a significant reduction of the pressure measured while 200 mM of sodium chloride (about 1% w_{sodium chloride}/w_{composition of lactase}) contributes to a far less significant reduction of pressure.

Therefore, citrate is a better solution to improve the filtrability of a formulation or composition comprising a lactase or lactase product than chloride. Preferably, sodium citrate is a better solution than sodium chloride. Additionally, similar results are expected for any lactase, in particular for any lactase from Bifidobacterium, including the lactases herein disclosed which are from Bifidobacterium.

### Example 2

Example 2 was carried out as Example 1 with the difference that several concentrations of sodium citrate were tested—Table 2.

TABLE 2
Sample 2 submitted to a stress period of 37° C. for 24 h.

| | | | Pressure remaining | Pressure |
|---|---|---|---|---|
| | | | in the system | reduced by the |
| Sample | | | vs control in % | composition in % |
| 2 | Control | 0 mM | 100 | — |
| | Na₃citrate | 2 mM | 64 | 36 |
| | | 10 mM | 49 | 51 |
| | | 20 mM | 27 | 73 |
| | | 100 mM | 30 | 70 |

Table 2 shows a decrease in the pressure rate measured when a composition comprising lactase and sodium citrate is filtered, regardless of the concentration of citrate, preferably sodium citrate used. Additionally, the results obtained with 10 mM of sodium citrate and shown in Tables 1 and 2 are in line, both showing that the pressure remaining in the filtration was of about 41 to 49%. Therefore, the pressure in the system was reduction by more than 50% versus the control, which shows that different samples of a lactase or lactase product showed an improvement in the filterability of the lactase or lactase product. Additionally, similar results are expected for any lactase, in particular for any lactase from Bifidobacterium, including the lactases herein disclosed which are from Bifidobacterium.

### Example 3

Example 3 was carried out as Example 1 with the exception that different anions were tested—Table 3.

TABLE 3
Samples 2 and 3 submitted to a stress period of 37° C. for 24 h.

| | | | Pressure remaining | Pressure |
|---|---|---|---|---|
| | | | in the system | reduced by the |
| Sample | | | vs control in % | composition in % |
| 2 | Control | 0 mM | 100 | — |
| | K₃citrate | 10 mM | 33 | 67 |
| | Kgluconate | 10 mM | 86 | 14 |
| | K₂-EDTA | 10 mM | 29 | 71 |
| 3 | Control | 0 mM | 100 | — |
| | Na₃citrate | 10 mM | 44 | 56 |
| | K₃citrate | 10 mM | 40 | 60 |
| | Na₂tartrate | 10 mM | 50 | 50 |
| | Na₂malate | 10 mM | 49 | 51 |

Table 3 confirms the results obtained with 10 mM of sodium citrate and shown in Tables 1 and 2. In other words, when 10 mM of sodium citrate is used it is possible to reduce more than 50% the pressure of the filtering system. This observation was consistently made using 3 different samples, thereby showing this effect is sample unrelated.

Moreover, a reduction of pressure (and therefore an improvement in the filterability) is also observed when potassium citrate is used, instead of sodium citrate, thereby illustrating the effect of citrate in improving the filterability of a lactase or lactase product. Preferably, 10 mM of potassium citrate is responsible for a reduction of pressure and as a consequence an improvement of filterability similarly to 10 mM of sodium citrate. Therefore, Table 3 shows the effect of citrate in improvement filterability regardless of the cation used (potassium or sodium).

Table 3 also shows an improvement of the pressure when anions other than citrate are used in the formulation or composition. The improvement is exemplified when gluconate, tartrate, malate, or ethylenediaminetetraacetate is added to the lactase or lactase product. Preferably, the improvement is exemplified by gluconate. More preferably, the improvement is exemplified by tartrate or malate, which lead to a reduction of pressure of about 50%. Even more preferably, the improvement is exemplified by ethylenediaminetetraacetate. When ethylenediaminetetraacetate is used, the pressure remaining in the filtration system is of about 29%, which means a decrease in pressure of about 71%. All values are given versus the control. Preferably, the improvement is exemplified by potassium gluconate. More preferably, the improvement is exemplified by sodium tartrate or sodium malate. Even more preferably, the improvement is exemplified by ethylenediaminetetraacetate disodium. All values are given versus the control.

Therefore, the effect observed for citrate in Tables 1-3 is extended to other anions, such as malate, tartrate, gluconate or ethylenediaminetetraacetate. Preferably, the improvement of filterability is extended to potassium citrate, sodium tartrate, sodium malate, potassium gluconate or ethylenediaminetetraacetate disodium, independently of the lactase sample tested. Additionally, similar results are expected for any lactase, in particular for any lactase from Bifidobacterium, including the lactases herein disclosed which are from Bifidobacterium.

### Example 4

Example 4 was carried out as Example 1 with the exception that the lactase prototypes were prepared from an unstandardized sample of NOLA® Fit from Chr. Hansen A/S, diluted in 50% glycerol in tap water (+sodium citrate) down to 5500 BLU/g strength or 2100 BLU/g strength. The lactase was exposed to a stress period of 6 weeks at 5° C. (Table 4) or 6 weeks at 5° C. followed by 37° C. incubation for 24 hours (Table 5). A stress period of 6 weeks at 5° C. is one of the possible representations of a storage condition.

TABLE 4
Sample 4 submitted to a stress period of 6 weeks at 5° C.

| | | | Pressure remaining | Pressure |
|---|---|---|---|---|
| | | | in the system | reduced by the |
| | | | vs control in % | composition in % |
| 5500 | Control | 0 mM | 100 | — |
| BLU/g | Na₃citrate | 10 mM | 44 | 56 |
| 2100 | Control | 0 mM | 100 | — |
| BLU/g | Na₃citrate | 10 mM | 50 | 50 |

TABLE 5
Sample 4 submitted to a stress period of 6 weeks at 5° C.
followed by 37° C. for 24 hours prior to filtration.

| | | | Pressure remaining | Pressure |
|---|---|---|---|---|
| | | | in the system | reduced by the |
| | | | vs control in % | composition in % |
| 5500 | Control | 0 mM | 100 | — |
| BLU/g | Na₃citrate | 10 mM | 64 | 36 |
| 2100 | Control | 0 mM | 100 | — |
| BLU/g | Na₃citrate | 10 mM | 39 | 61 |

Both tables (Tables 4 and 5) show the relevance of supplementing a formulation or composition comprising a lactase or lactase product with an anion such as citrate which is then placed under a representative storage condition, as even after a storage period, the formulation of composition retains the improved filterability characteristics as the pressure remaining in the system remains less than the pressure of the control when citrate is used or supplemented to the formulation or composition comprising a lactase or lactase product. Additionally, the effect observed is independent of the enzyme dosage or strength used, as for both dosages there is less pressure retained in the system. All values are given versus the control. Therefore, Tables 4 and 5 show the effect of the anion is not time-limited or enzyme strength-related. Additionally, this effect is also expected (based on the previous examples) to be seem for the remaining anions: malate, tartrate, gluconate, ethylenediaminetetraacetate, and/or combinations thereof. Finally, similar results are expected for any lactase, in particular for any lactase from Bifidobacterium, including the lactases herein disclosed which are from Bifidobacterium.

### Example 5

The lactase from Bifidobacterium, in particular SEQ ID NO: 40 from Bifidobacterium longum, was also tested—sample 5. An unstandardized concentrate sample was diluted in 50% glycerol in tap water (+sodium citrate or sodium tartrate) down to 14000 BLU/g strength. In this example, the strength of the lactase used was determined by comparing the activity of the lactase versus the activity of a lactase with a well-known strength, which may be, for example, the same lactase used in Examples 1-4. Alternatively, the strength may also be determined as explained in Examples 16 and 17 of the WO2018189224 or as in EP 3 568 023 multiplied by a factor of 2. The resulting 14000 prototypes were pre-filtered through a 0.45 μm VWR Media Bottle top filtration unit, PES 0.45 μm, 90 mm (Cat no #514-0300), and stored at 5° C. The samples were brought to room temperature for 1 h before the filterability test. The samples are filtered using a SartoFLOW Smart filtration unit, through a 0.45 μm pre-filter (Sartorius Syringe Filter, Minisart NML, CA, 28 mm, 0.45 μm, sterile, Cat #16555-K) and a 0.2 μm sterile filter (Pall Life Science Acrodisc 25 mm Syringe Filter 0.2 μm Fluorodyne II membrane (sterile) (VWR cat: 516-8973P) in sequence and pre-wetted with MilliQ Water (ELGA Purelab flex). The flow rate was kept stable at 6.5 g/min (+/−10%), and the initial rate of increase in back pressure on the system was measured and calculated in bar/hour, through a linear fit. The increase in back pressure rate is used to evaluate filterability of the samples, meaning that samples that result in higher pressure increase rates have lower filterability.

TABLE 6
Sample 5 submitted to a stress period of 5° C. for 4 days.

| | | | Pressure remaining | Pressure |
|---|---|---|---|---|
| | | | in the system | reduced by the |
| Sample | | | vs control in % | composition in % |
| 5 | Control | 0 mM | 100 | — |
| | Na₃citrate | 20 mM | 78 | 22 |
| | Na₂tartrate | 20 mM | 60 | 40 |

Table 6 shows a reduction in the pressure when filtering sample 5 and therefore shows an improvement of the filterability of a lactase sample when citrate or tartrate is used. Additionally, the combination of Example 5 with the remaining examples herein disclosed show that a reduction of the pressure of the filtration system occurs when malate, tartrate, citrate, gluconate, ethylenediaminetetraacetate, or a combination thereof are used in a formulation or composition of a lactase or lactase product regardless of the lactase used. Thus, Example 5 confirms that similar results are obtained regardless of the lactase or lactase product used, preferably similar results are obtained for any lactase from Bifidobacterium, including the lactases herein disclosed which are from Bifidobacterium, more preferably similar results are obtained for any lactase from Bifidobacterium bifidum or longum, as exemplified in Examples 1-5.

### Example 6

The lactase from Kluyveromyces, in particular SEQ ID NO: 53 from Kluyveromyces lactis, was also tested—sample 6. This lactase is also known as HA-Lactase® from Chr. Hansen A/S. HA-Lactase® 5200 batch (from Chr. Hansen A/S) was diluted to a 50% (v/v) glycerol in tap water (control) or supplemented with an anion selected from tartrate and citrate, or a combination thereof, such that a final strength of 2600 BLU/g strength was reached. In this example, the strength of the lactase used was determined by comparing the activity of the lactase versus the activity of a lactase with a well-known strength, which may be, for example, the same lactase used in Examples 1-4. Alternatively, the strength may also be determined as explained in Examples 16 and 17 of the WO2018189224. The resulting 2600 prototypes were pre-filtered through a 0.45 μm VWR Media Bottle top filtration unit, PES 0.45 μm, 90 mm (Cat no #514-0300), and stored at 5° C. After incubation at 37° C. for 24 h, the samples are brought to room temperature for 1 h before the filterability test. Preferably the samples were brought to a temperature ranging from 15 to 30° C., preferably 15 to 25° C., more preferably 20 to 25° C. (at 1 atm). The samples are filtered using a SartoFLOW Smart filtration unit, through a 0.45 μm pre-filter (Sartorius Syringe Filter, Minisart NML, CA, 28 mm, 0.45 μm, sterile, Cat #16555-K) and a 0.2 μm sterile filter (Pall Life Science Acrodisc 25 mm Syringe Filter 0.2 μm Fluorodyne II membrane (sterile) (VWR cat: 516-8973P) in sequence and pre-wetted with MilliQ Water (ELGA Purelab flex). The flow rate was kept stable at 6.5 g/min (+/−10%), and the initial rate of increase in back pressure on the system was measured and calculated in bar/hour, through a linear fit. The increase in back pressure rate is used to evaluate filterability of the samples, meaning that samples that result in higher pressure increase rates have lower filterability.

TABLE 7
Sample 6 submitted to a stress period of 37° C. for 24 h.

| | | | Pressure remaining | Pressure |
|---|---|---|---|---|
| | | | in the system | reduced by the |
| Sample | | | vs control in % | composition in % |
| 6 | Control | 0 mM | 100 | — |
| | Na₃citrate | 20 mM | 3 | 97 |
| | Na₂tartrate | 20 mM | 13 | 87 |

Table 7 shows a reduction in the pressure when filtering sample 6 and therefore shows an improvement of the filterability of a lactase sample when citrate or tartrate is used. Additionally, the combination of Example 6 with the remaining examples herein disclosed show that a reduction of the pressure of the filtration system occurs when malate, tartrate, citrate, gluconate, ethylenediaminetetraacetate, or a combination thereof are used in a formulation or composition of a lactase or lactase product regardless of the lactase used. Therefore, Example 6 confirms that similar results are obtained regardless of the lactase or lactase product used, preferably similar results are obtained for any lactase from Bifidobacterium or Kluyveromyces, including the lactases herein disclosed which are from both these genera, more preferably similar results are obtained for any lactase from Bifidobacterium bifidum or Bifidobacterium longum or Kluyveromyces lactis.

In conclusion, the filterability of a lactase sample can be improved provided that malate, tartrate, citrate, gluconate, ethylenediaminetetraacetate, or a combination thereof, is part of the formulation or composition comprising the lactase or lactase product prior to the filtration step. The cation may be sodium or potassium.

### NUMBERED EMBODIMENTS

### Composition

Embodiment 1. Composition comprising a lactase and an anion selected from malate, tartrate, citrate, gluconate, ethylenediaminetetraacetate, and/or a combination thereof.

Embodiment 2. Composition according to the previous embodiment, wherein the anion is 0.1-250 mM of malate, tartrate, citrate, gluconate, ethylenediaminetetraacetate, and/or a combination thereof; preferably 1-150 mM of malate, tartrate, citrate, gluconate, ethylenediaminetetraacetate, and/or a combination thereof, more preferably 2-100 mM of malate, tartrate, citrate, gluconate, ethylenediaminetetraacetate, and/or a combination thereof, even more preferably 10-20 mM of malate, tartrate, citrate, gluconate, ethylenediaminetetraacetate, and/or a combination thereof.

Embodiment 3. Composition according to any of the previous embodiments 1-2, wherein the lactase is a neutral lactase or a acidic lactase.

Embodiment 4. Composition according to any of the previous embodiments 1-3, wherein the lactase has an amino acid sequence which is at least 75% identical to any of SEQ ID NO: 1-53, preferably at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53. The most preferred sequences are SEQ ID NO: 1, 7, 40, 52, 53 and any sequence having 75%-100% or 80%-100% or 85%-100% identical to any of SEQ ID NO: 1, 7, 40, 52, 53, such as 86%, 87%, 88%, 89%, 90%, 91%, 92%, 93%, 94%, 95%, 96%, 97%, 98%, 99%, or 100% identical to any of SEQ ID NO: 1, 7, 40, 52, 53.

Embodiment 5. Composition according to any of previous embodiments 1-4 comprising a polyol selected among glycerol, sorbitol, monopropylene glycol, sucrose, glucose, galactose or lactose, or a combination thereof, preferably glycerol.

Embodiment 6. Composition according to the previous embodiment 5, wherein the polyol is at a concentration of 10-70% w_{polyol}/w_{composition}, preferably 20-80% w_{polyol}/w_{composition}, more preferably 30-60% w_{polyol}/w_{composition}.

Embodiment 7. Composition according to any of the previous embodiments 1-6, wherein the composition is a liquid lactase composition.

Embodiment 8. Composition according to any of the previous embodiments 1-7, wherein the composition is a sterile liquid lactase composition.

Embodiment 9. Sterile-filtered liquid lactase composition comprising: a lactase;
an anion selected from a list consisting of malate, tartrate, citrate, gluconate, and ethylenediaminetetraacetate;
and preferably with a cation selected from a list consisting of sodium and potassium.

Embodiment 10. Sterile-filtered liquid lactase composition according to embodiment 9, wherein the anion is selected a list consisting of 0.1-250 mM of malate, tartrate, citrate, gluconate, and ethylenediaminetetraacetate, preferably 1-150 mM of malate, tartrate, citrate, gluconate, and ethylenediaminetetraacetate, more preferably 2-100 mM of malate, tartrate, citrate, gluconate, and ethylenediaminetetraacetate, even more preferably 10-20 mM of malate, tartrate, citrate, gluconate, and ethylenediaminetetraacetate.

Embodiment 11. Sterile-filtered liquid lactase composition according to any of the previous embodiments 9-11, wherein the anion and the cation form a salt selected from a list consisting of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt; preferably wherein the salt is selected from a list consisting of 0.1-250 mM of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt; more preferably 1-150 mM sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt, even more preferably 2-100 mM sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt or 10-20 mM sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt.

Embodiment 12. Sterile-filtered liquid lactase composition according to any of previous embodiments 9-11, wherein the lactase is a neutral lactase or an acidic lactase.

Embodiment 13. Sterile-filtered liquid lactase composition according to any of embodiments 9-12 wherein the lactase has an amino acid sequence which is at least 75% identical to any of SEQ ID NO: 1-53, preferably at least 85% identical to any of SEQ ID NO: 1-53; more preferably at least 90% identical to any of SEQ ID NO: 1-53, preferably at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53. The most preferred sequences are SEQ ID NO: 1, 7, 40, 52, 53 and any sequence having 75%-100% or 80%-100% or 85%-100% identical to any of SEQ ID NO: 1, 7, 40, 52, 53, such as 86%, 87%, 88%, 89%, 90%, 91%, 92%, 93%, 94%, 95%, 96%, 97%, 98%, 99%, or 100% identical to any of SEQ ID NO: 1, 7, 40, 52, 53.

Embodiment 14. Sterile-filtered liquid lactase composition according to any of embodiments 9-13 comprising a polyol selected among glycerol, sorbitol, monopropylene glycol, sucrose, glucose, galactose, lactose, and/or a combination thereof, preferably glycerol

Embodiment 15. Sterile-filtered liquid lactase composition according to embodiment 14, wherein the polyol is at a concentration of 10-70% w_{polyol}/w_{composition}, preferably 20-80% w_{polyol}/w_{composition}, more preferably 30-60% w_{polyol}/w_{composition}.

### Uses

Embodiment 16. Use of an anion selected from malate, tartrate, citrate, gluconate, ethylenediaminetetraacetate, and/or a combination thereof, for improving filterability of a composition according to any of the previous embodiments 1-8. Therefore, said anion is used as an improver of the filterability of a composition as herein disclosed.

Embodiment 17. Use of an anion selected from malate, tartrate, citrate, gluconate, ethylenediaminetetraacetate, and/or a combination thereof, for reducing pressure of a filtration system of a composition according to any of the previous embodiments 1-8. Therefore, said anion is used as an reducing pressure element or agent of a filtration system, which is used for filtering a composition as herein disclosed.

Embodiment 18. Use of an anion selected from malate, tartrate, citrate, gluconate, ethylenediaminetetraacetate, and/or a combination thereof, for reducing pressure of an in-line filtration system of a production process of a dairy product, when a composition as the one herein disclosed (any of the previous embodiments 1-8) is used in the product process of the dairy product. Therefore, said anion is used as an reducing pressure element or agent of in-line filtration system, which is used for filtering a composition as herein disclosed.

Embodiment 19. Use according to any of the previous embodiments 16-18, wherein the anion is 0.1-250 mM of malate, tartrate, citrate, gluconate, ethylenediaminetetraacetate, and/or a combination thereof, preferably 1-150 mM of malate, tartrate, citrate, gluconate, ethylenediaminetetraacetate, and/or a combination thereof, more preferably 2-100 mM of malate, tartrate, citrate, gluconate, ethylenediaminetetraacetate, and/or a combination thereof, even more preferably 10-20 mM of malate, tartrate, citrate, gluconate, ethylenediaminetetraacetate, and/or a combination thereof.

Embodiment 20. Use of an anion selected a list consisting of malate, tartrate, citrate, gluconate, and ethylenediaminetetraacetate, for improving filterability of a composition, and/or for reducing pressure of a filtration system of a composition, preferably for reducing pressure of an in-line filtration system of a production process of a dairy product, wherein the composition comprises a lactase and the anion. Therefore, said anion is used as an improver of the filterability of a composition as herein disclosed and/or said anion is used as an reducing pressure element or agent of a filtration system, which is used for filtering a composition as herein disclosed, preferably said anion is used as an reducing pressure element or agent of in-line filtration system, which is used for filtering a composition as herein disclosed.

Embodiment 21. Use according to embodiment 20, wherein the anion is selected from a list consisting of 0.1-250 mM of malate, tartrate, citrate, gluconate, and ethylenediaminetetraacetate, preferably 1-150 mM of malate, tartrate, citrate, gluconate, and ethylenediaminetetraacetate, more preferably 2-100 mM of malate, tartrate, citrate, gluconate, and ethylenediaminetetraacetate, even more preferably 10-20 mM of malate, tartrate, citrate, gluconate, and ethylenediaminetetraacetate.

Embodiment 22. Use according to any of embodiments 20-21, wherein the composition comprises a cation selected from a list consisting of sodium and potassium.

Embodiment 23. Use according to any of embodiments 20-22, wherein the anion and the cation form a salt selected from a list consisting of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt.

Embodiment 24. Use according to any of the previous embodiments 20-23, wherein the salt is selected from a list consisting of 0.1-250 mM of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt; preferably 1-150 mM of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt; more preferably 2-100 mM of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt; even more preferably 10-20 mM of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt.

Embodiment 25. Use according to any of embodiments 20-24, wherein the lactase is a neutral lactase or an acidic lactase.

Embodiment 26. Use according to any of embodiments 20-25, wherein the lactase has an amino acid sequence which is at least 75% identical to any of SEQ ID NO: 1-53, preferably at least 85% identical to any of SEQ ID NO: 1-53; more preferably at least 90% identical to any of SEQ ID NO: 1-53, preferably at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53. The most preferred sequences are SEQ ID NO: 1, 7, 40, 52, 53 and any sequence having 75%-100% or 80%-100% or 85%-100% identical to any of SEQ ID NO: 1, 7, 40, 52, 53, such as 86%, 87%, 88%, 89%, 90%, 91%, 92%, 93%, 94%, 95%, 96%, 97%, 98%, 99%, or 100% identical to any of SEQ ID NO: 1, 7, 40, 52, 53.

Embodiment 27. Use according to any of embodiments 20-26, wherein the composition comprises a polyol selected among glycerol, sorbitol, monopropylene glycol, sucrose, glucose, galactose or lactose, or combination thereof, preferably glycerol; more preferably wherein the polyol is at a concentration of 10-70% w_{polyol}/w_{composition}, preferably 20-80% w_{polyol}/w_{composition}, more preferably 30-60% w_{polyol}/w_{composition}.

Embodiment 28. Use according to any of embodiments 20-27, wherein the composition is a liquid lactase composition or a sterile liquid lactase composition or a sterile-filtered liquid lactase composition.

### Methods

Embodiment 29. Method for reducing pressure in a filtration system comprising the step of filtrating a lactase composition according to any of the previous embodiments 1-8.

Embodiment 30. Method according to the previous embodiment 29, wherein the filtration system is an in-line filtration system of a production process of a dairy product.

Embodiment 31. Method according to any of the previous embodiments 29-30, wherein the filtration system is an aseptic in-line dosing system.

Embodiment 32. Method for reducing pressure in a filtration system comprising a step of filtrating a composition in said filtration system, wherein the composition comprises a lactase and an anion selected from a list consisting of malate, tartrate, citrate, gluconate, and ethylenediaminetetraacetate.

Embodiment 33. Method according to embodiment 32, wherein the anion is selected from a list consisting of 0.1-250 mM of malate, tartrate, citrate, gluconate, ethylenediaminetetraacetate, and/or a combination thereof; preferably 1-150 mM of malate, tartrate, citrate, gluconate, ethylenediaminetetraacetate, and/or a combination thereof; more preferably 2-100 mM of malate, tartrate, citrate, gluconate, ethylenediaminetetraacetate, and/or a combination thereof, even more preferably 10-20 mM of malate, tartrate, citrate, gluconate, ethylenediaminetetraacetate, and/or a combination thereof.

Embodiment 34. Method according to any embodiments 32-33, comprising a cation selected from sodium and potassium.

Embodiment 35. Method according to any embodiments 32-34, wherein the anion and the cation form a salt selected a list consisting of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt.

Embodiments 36. Method according to any of the previous embodiments 32-35, wherein the salt is selected from a list consisting of 0.1-250 mM, preferably 1-150 mM, more preferably 2-100 mM, even more preferably 10-20 mM, of sodium malate, potassium malate, sodium tartrate, potassium tartrate, sodium citrate, potassium citrate, sodium gluconate, potassium gluconate, ethylenediaminetetraacetate disodium salt, and ethylenediaminetetraacetate dipotassium salt.

Embodiments 37. Method according to any of embodiments 32-36, wherein the lactase is a neutral lactase or an acidic lactase.

Embodiments 38. Method according to any of embodiments 32-37, wherein the lactase has an amino acid sequence which is at least 75% identical to any of SEQ ID NO: 1-53, preferably at least 85% identical to any of SEQ ID NO: 1-53; more preferably at least 90% identical to any of SEQ ID NO: 1-53, preferably at least 80%, or at least 85%, 86%, 87%, 88%, 89%, 90%, 91%, 92%, 93%, 94%, 95%, 96%, 97%, 98%, 99%, or 100% identical to any of SEQ ID NO: 1-53. More preferably, said lactase may have an amino acid sequence which is 75%-100% or 80%-100% or 85%-100% identical to any of SEQ ID NO: 1-53, such as 86%, 87%, 88%, 89%, 90%, 91%, 92%, 93%, 94%, 95%, 96%, 97%, 98%, 99%, or 100% identical to any of SEQ ID NO: 1-53. Even more preferably, 90%-100% identical to any of SEQ ID NO: 1-53, such as 91%, 92%, 93%, 94%, 95%, 96%, 97%, 98%, 99%, or 100% identical to any of SEQ ID NO: 1-53. The most preferred sequences are SEQ ID NO: 1, 7, 40, 52, 53 and any sequence having 75%-100% or 80%-100% or 85%-100% identical to any of SEQ ID NO: 1, 7, 40, 52, 53, such as 86%, 87%, 88%, 89%, 90%, 91%, 92%, 93%, 94%, 95%, 96%, 97%, 98%, 99%, or 100% identical to any of SEQ ID NO: 1, 7, 40, 52, 53.

Embodiments 39. Method according to any of embodiments 32-38, wherein the composition comprises a polyol selected among glycerol, sorbitol, monopropylene glycol, sucrose, glucose, galactose or lactose, or a combination thereof, preferably glycerol; more preferably wherein the polyol is at a concentration of 10-70% w_{polyol}/w_{composition}, preferably 20-80% w_{polyol}/w_{composition}, more preferably 30-60% w_{polyol}/w_{composition}.

Embodiments 40. Method according to any of embodiments 32-39, wherein the composition is a liquid lactase composition or a sterile liquid lactase composition or a sterile-filtered liquid lactase composition.

Embodiments 41. Method according to any of embodiments 32-40, wherein the filtration system is an in-line filtration system of a production process of a dairy product.

Embodiments 42. Method according to any of embodiments 32-41, wherein the filtration system is an aseptic in-line dosing system.

1. A sterile-filtered liquid lactase composition, comprising:
a lactase;
an anion selected from a list consisting of malate, tartrate, citrate, gluconate, and ethylenediaminetetraacetate; and
a cation selected from a list consisting of sodium and potassium.

2. The composition of claim 1, wherein the composition comprises 0.1-250 mM of the anion.

3. The composition of claim 1, wherein the lactase is a neutral lactase or an acidic lactase and has an amino acid sequence which is at least 75% identical to any of SEQ ID NO: 1-53.

4. The composition of claim 1, further comprising a polyol selected from a list consisting of: glycerol, sorbitol, monopropylene glycol, sucrose, glucose, galactose, lactose, and/or a combination thereof.

5-10. (canceled)

11. A method for reducing pressure in a filtration system comprising:
filtrating a composition in said filtration system,
wherein the composition comprises a lactase and an anion selected from a list consisting of malate, tartrate, citrate, gluconate, and ethylenediaminetetraacetate.

12. The method of claim 11, wherein the anion is in a concentration of 0.1-250 mM.

13. The method of claim 11, wherein the composition comprises a cation selected from the list consisting of sodium and potassium.

14. The method of claim 11, wherein the lactase is a neutral lactase or an acidic lactase and has an amino acid sequence which is at least 75% identical to any of SEQ ID NO: 1-53.

15. The method of claim 11, wherein the composition further comprises a polyol selected among glycerol, sorbitol, monopropylene glycol, sucrose, glucose, galactose or lactose, or a combination thereof.

16. The method of claim 11, wherein the composition is a liquid lactase composition or a sterile liquid lactase composition or a sterile-filtered liquid lactase composition.

17. The method of claim 11, wherein the filtration system is an in-line filtration system of a production process of a dairy product.

18. The method of claim 11, wherein the filtration system is an aseptic in-line dosing system.

19. The composition of claim 4, wherein the composition comprises a polyol at concentration of 10-70% w_{polyol}/w_{composition}.

20. The composition of claim 1, wherein the composition comprises 1-150 mM of the anion.

21. The composition of claim 1, wherein the composition comprises 10-20 mM, of the anion.

22. The composition of claim 1, wherein the anion is selected from the group comprising malate, tartrate, citrate, gluconate, and ethylenediaminetetraacetate.

23. The composition of claim 3, wherein the lactase is a neutral lactase or an acidic lactase having an amino acid sequence that is at least 85% identical to any of SEQ ID NO:
1-53.

24. The composition of claim 3, wherein the lactase is a neutral lactase or an acidic lactase having an amino acid sequence that is at least 90% identical to any of SEQ ID NO:
1-53.

25. The composition of claim 1, further comprising a polyol selected from a list consisting of: glycerol, sorbitol, monopropylene glycol, sucrose, glucose, galactose, lactose, and/or a combination thereof, wherein the polyol is at a concentration of 20-80% w_{polyol}/w_{composition}.

26. The composition of claim 1, further comprising a polyol selected from a list consisting of: glycerol, sorbitol, monopropylene glycol, sucrose, glucose, galactose, lactose, and/or a combination thereof, wherein the polyol is at a concentration of 30-60% w_{polyol}/w_{composition}.
